(19) United States
(12) Patent Application Publication
(10) Pub. No.: US 2017/0312153 A1
(43) Pub. Date: Nov. 2, 2017

(54) PATIENT LIFT ORTHOSIS

(71) Applicant: EXORISE, L.L.C., Silver Spring, MD (US)

(72) Inventors: Scott M. PAUL, Silver Spring, MD (US); Kevin Brian MOON, Redmond, WA (US); Brooke Michele MAHOSKI, Oyster Bay, NY (US); Rowan Patrick CADE, Shaker Heights, OH (US); Thomas Clifford Brunoski BERNSTEIN, Ney York, NY (US); Emily Jane MCNALLY, Baltimore, MD (US); Alexis Kayla SCHOLTZ, South Pasadena, CA (US); Sue Min CHO, Youngin-si (KR); Zhou LI, Dalian (CN); Baxter Rhodes ELDRIDGE, Danville, CA (US); Mary Theresa NAHILL, Winchester, MA (US); Susanna Elaine KELLOGG, Norfolk, VA (US); Ronann Harl Carrero ORTIZ, Milton, FL (US); Matthew Joseph GRAMUGLIA, East Brunswick, NJ (US)

(73) Assignee: EXORISE, L.L.C., Silver Spring, MD (US)

(21) Appl. No.: 15/583,254

(22) Filed: May 1, 2017

Related U.S. Application Data
(60) Provisional application No. 62/330,477, filed on May 2, 2016.

(51) Int. Cl. A61G 5/14; A61F 5/01

(52) U.S. Cl. CPC A61G 5/14; A61F 5/0123; A61G 2203/34; A61G 2203/10; A61G 2203/70; A61G 2203/30

(57) ABSTRACT

A patient lift orthosis system including an input controller and at least one orthosis. The at least one orthosis including an upper orthosis assembly and a lower orthosis assembly rotatably attached to the upper orthosis assembly via a joint. The at least one orthosis further including an output controller attached to at least one of the upper orthosis assembly, the lower orthosis assembly, and the joint, the output controller being in communication with the input controller. The at least one orthosis further including an actuator attached to at least one of the upper orthosis assembly, the lower orthosis assembly, and the joint, the actuator being in communication with the output controller. The output controller is configured to actuate the actuator to rotate one of the upper orthosis assembly and the lower orthosis assembly about the joint based upon an input communicated from the input controller.

### CROSS REFERENCE TO RELATED APPLICATIONS

The present application claims the benefit of U.S. Provisional Application No. 62/330,477 filed on May 2, 2016, the content of which is expressly incorporated herein by reference in its entirety.

### FIELD OF THE DISCLOSURE

The technology relates to the general field of medical devices, and has certain specific application to patient assistive devices.

### BACKGROUND OF THE DISCLOSURE

Healthcare workers, particularly nurses and nursing assistants, endure some of the highest rates of occupational musculoskeletal injury and the most missed workdays due to these injuries. Patient-handling events, including lifting, repositioning, and transferring, result in a third of the total number of lost workdays. Hospitals face significant monetary ramifications that arise from these injuries. Considering only neck, back, and shoulder injuries among healthcare workers (e.g., nursing assistants, registered nurses, licensed practical nurses, occupational therapists, physical therapists therapy aides, etc.), hospitals incur significant annual expenses on these healthcare workers' compensation, lawsuits, and medical bills. Accordingly, there exists a need for improved systems and methods for patient-handling events, including lifting, reposition, and transferring.

### SUMMARY OF THE EMBODIMENTS OF THE DISCLOSURE

Aspects of the present disclosure are directed to a patient lift orthosis system comprising an input controller, and at least one orthosis, the at least one orthosis including an upper orthosis assembly, a lower orthosis assembly rotatably attached to the upper orthosis assembly via a joint, an output controller attached to at least one of the upper orthosis assembly, the lower orthosis assembly, and the joint, the output controller being in communication with the input controller, and an actuator attached to at least one of the upper orthosis assembly, the lower orthosis assembly, and the joint, the actuator being in communication with the output controller. The output controller is configured to actuate the actuator to rotate one of the upper orthosis assembly and the lower orthosis assembly about the joint based upon an input communicated from the input controller.

In embodiments, the patient lift orthosis system further comprises at least one sensor configured to provide sensory feedback. The at least one sensor is attached to at least one of the upper orthosis assembly, the lower orthosis assembly, and the joint. The at least one sensor being in communication with the output controller. The output controller is configured to actuate the actuator to rotate at least one of the upper orthosis assembly and the lower orthosis assembly about the joint based upon the input communicated from the input controller and the sensory feedback communicated from the at least one sensor.

In additional embodiments, the patient lift orthosis system further comprises a plurality of straps configured to hold a leg of a patient against the at least one orthosis. At least a first strap of the plurality straps is attached to the upper orthosis assembly, and at least a second strap of the plurality of straps is attached to the lower orthosis assembly.

In some embodiments, the patient lift orthosis system further comprises at least one battery attached to at least one of the upper orthosis assembly and the lower orthosis assembly. The at least one battery is connected to the actuator and is configured to deliver power sufficient to rotate the one of the upper orthosis assembly and the lower orthosis assembly about the joint when the orthosis is attached to a patient.

In certain embodiments, the actuator is fixedly attached to the lower orthosis assembly and rotatably attached to the upper orthosis assembly such that the actuator is configured to rotate the upper orthosis assembly about the joint relative to the lower orthosis assembly.

In yet further embodiments, the at least one battery and the output controller are fixed to the lower orthosis assembly.

In further embodiments, the upper orthosis assembly and the lower orthosis assembly each include at least one cuff configured to support a leg of a patient.

In additional embodiments, the patient lift orthosis system further comprises a knee cuff that is rotatably attached to the orthosis and that is configured to rotate independently from the upper orthosis assembly and the lower orthosis assembly.

In some embodiments, the at least one cuff of the upper orthosis assembly, the at least one cuff of the lower orthosis assembly, and the knee cuff are each at least one of adjustable and replaceable.

In certain embodiments, the at least one cuff of the upper orthosis assembly, the at least one cuff of the lower orthosis assembly, and the knee cuff each define a concave curved shape configured to receive portions of the leg of the patient.

In yet further embodiments, the actuator is an electric motor.

In further embodiments, the orthosis further includes a harmonic drive gearbox attached to the electric motor.

In additional embodiments, the orthosis further includes a high ratio gearbox coupled to the electric motor and a bevel gearbox coupled to the high ratio gearbox via a clutch mechanism.

In certain embodiments, the actuator is a linear actuator.

Additional aspects of the present disclosure are directed to a patient lift orthosis system comprising an input controller, and a first orthosis and a second orthosis. The first and the second orthoses each including an upper orthosis assembly, a lower orthosis assembly rotatably attached to the upper orthosis assembly via a joint, an output controller attached to at least one of the upper orthosis assembly, the lower orthosis assembly, and the joint, the output controller being in communication with the input controller, and an actuator attached to at least one of the upper orthosis assembly, the lower orthosis assembly, and the joint, the actuator being in communication with the output controller. The first orthosis being configured to be attached to a first limb of a patient and the second orthosis being configured to be attached to a second limb of the patient. For each of the first and the second orthoses respectively, the output controller is configured to actuate the actuator to rotate one of the upper orthosis assembly and the lower orthosis assembly about the joint based upon an input communicated from the input controller.

In yet further embodiments, the output controller of the first orthosis and the output controller of the second orthosis are configured to communicate to each other.

In further embodiments, the first orthosis and the second orthosis each further include at least one sensor configured to provide sensory feedback, the at least one sensor is attached to at least one of the upper orthosis assembly, the lower orthosis assembly, and the joint, the at least one sensor being in communication with the output controller. For each of the first and the second orthoses respectively, the output controller is configured to actuate the actuator to rotate one of the upper orthosis assembly and the lower orthosis assembly about the joint based upon the input communicated from the input controller and the sensory feedback communicated from the at least one sensor.

Additional aspects of the present disclosure are directed to a method for lifting a patient using a patient lift orthosis system comprising an input controller and at least one orthosis. The at least one orthosis including an upper orthosis assembly, a lower orthosis assembly rotatably attached to the upper orthosis assembly via a joint, an output controller attached to at least one of the upper orthosis assembly, the lower orthosis assembly, and the joint, the output controller being in communication with the input controller, and an actuator attached to at least one of the upper orthosis assembly, the lower orthosis assembly, and the joint, the actuator being in communication with the output controller. The method comprising positioning a thigh of the patient on the upper orthosis assembly and positioning a calf of the patient on the lower orthosis assembly, aligning the joint with a knee of the patient, activating the input controller to communicate an input to the output controller that actuates the actuator to rotate one of the upper orthosis assembly and the lower orthosis assembly about the joint to lift the patient from a seated position to a standing position.

In certain embodiments, the method of lifting the patient using the patient lift orthosis system further comprises locking the joint at the standing position.

In yet further embodiments, the method of lifting the patient using the patient lift orthosis system further comprises communicating from the input controller to the output controller to unlock the joint and to actuate the actuator to rotate the one of the upper orthosis assembly and the lower orthosis assembly about the joint to lower the patient from the standing position to the seated position.

### BRIEF DESCRIPTION OF THE DRAWINGS

The novel features which are characteristic of the systems, both as to structure and method of operation thereof, together with further aims and advantages thereof, will be understood from the following description, considered in connection with the accompanying drawings, in which embodiments of the system are illustrated by way of example. It is to be expressly understood, however, that the drawings are for the purpose of illustration and description only, and they are not intended as a definition of the limits of the system. For a more complete understanding of the disclosure, as well as other aims and further features thereof, reference may be had to the following detailed description of the disclosure in conjunction with the following exemplary and non-limiting drawings wherein:

FIGS. 1A-1C illustrate views of an exemplary patient lift orthosis system attached to a patient in accordance with aspects of the present disclosure;

FIG. 2 illustrates a schematic view of an exemplary control system for the patient lift orthosis in accordance with aspects of the present disclosure;

FIG. 3 illustrates a perspective view of an exemplary knee orthosis of the patient lift orthosis system in accordance with aspects of the present disclosure;

FIGS. 4A and 4B illustrate side views of an exemplary knee orthosis of the patient lift orthosis system in accordance with aspects of the present disclosure;

FIGS. 5A and 5B illustrate top views of an exemplary knee orthosis of the patient lift orthosis system in accordance with aspects of the present disclosure;

FIG. 6A illustrates an exploded perspective anterior view of an exemplary knee orthosis having an electric motor and a harmonic drive gearbox in accordance with aspects of the present disclosure;

FIG. 6B illustrates an exploded perspective posterior view of the exemplary knee orthosis of FIG. 6A in accordance with aspects of the present disclosure;

FIGS. 7A and 7B illustrate views of an exemplary knee orthosis having an electric motor, a high ratio gearbox and a bevel gearbox in accordance with aspects of the present disclosure;

FIG. 8 illustrates a side view of an exemplary knee orthosis having a linear actuator in accordance with aspects of the present disclosure;

FIGS. 9 illustrates a cut-away side view of an exemplary knee orthosis powered by a battery and charged by a charging station in accordance with aspects of the present disclosure;

FIG. 10 illustrates an exemplary process for assisting a patient to rise from a seated position to a standing position using the patient lift orthosis system; and

FIG. 11 is an exemplary system environment for use in accordance with the embodiments of control systems described herein.

### DETAILED DISCLOSURE

In the following description, the various embodiments of the present disclosure will be described with respect to the enclosed drawings. As required, detailed embodiments of the present disclosure are discussed herein; however, it is to be understood that the disclosed embodiments are merely exemplary of the embodiments of the disclosure that may be embodied in various and alternative forms. The figures are not necessarily to scale and some features may be exaggerated or minimized to show details of particular components. Therefore, specific structural and functional details disclosed herein are not to be interpreted as limiting, but merely as a representative basis for teaching one skilled in the art to variously employ the present disclosure.

The particulars shown herein are by way of example and for purposes of illustrative discussion of the embodiments of the present disclosure only and are presented in the cause of providing what is believed to be the most useful and readily understood description of the principles and conceptual aspects of the present disclosure. In this regard, no attempt is made to show structural details of the present disclosure in more detail than is necessary for the fundamental understanding of the present disclosure, such that the description, taken with the drawings, making apparent to those skilled in the art how the forms of the present disclosure may be embodied in practice.

As used herein, the singular forms “a,” “an,” and “the” include the plural reference unless the context clearly dictates otherwise. For example, reference to “an orthosis” would also indicate multiple orthoses can be present unless specifically excluded.

Except where otherwise indicated, all numbers expressing quantities used in the specification and claims are to be understood as being modified in all instances by the term “about.” Accordingly, unless indicated to the contrary, the numerical parameters set forth in the specification and claims are approximations that may vary depending upon the desired properties sought to be obtained by embodiments of the present disclosure. At the very least, and not to be considered as an attempt to limit the application of the doctrine of equivalents to the scope of the claims, each numerical parameter should be construed in light of the number of significant digits and ordinary rounding conventions (unless otherwise explicitly indicated).

Additionally, the recitation of numerical ranges within this specification is considered to be a disclosure of all numerical values and ranges within that range (unless otherwise explicitly indicated). For example, if a range is from about 1 to about 50, it is deemed to include, for example, 1, 7, 34, 46.1, 23.7, or any other value or range within the range.

As used herein, the terms “about” and “approximately” indicate that the amount or value in question may be the specific value designated or some other value in its neighborhood. Generally, the terms “about” and “approximately” denoting a certain value is intended to denote a range within ±5% of the value. As one example, the phrase “about 100” denotes a range of 100±5, i.e., the range from 95 to 105. Generally, when the terms “about” and “approximately” are used, it can be expected that similar results or effects according to the disclosure can be obtained within a range of ±5% of the indicated value.

As used herein, the term “and/or” indicates that either all or only one of the elements of said group may be present. For example, “A and/or B” indicates “only A, or only B, or both A and B.” In the case of “only A,” the term also covers the possibility that B is absent, i.e., “only A, but not B.”

The term “substantially parallel” refers to deviating less than 20° from parallel alignment and the term “substantially perpendicular” refers to deviating less than 20° from perpendicular alignment. The term “parallel” refers to deviating less than 5° from mathematically exact parallel alignment. Similarly “perpendicular” refers to deviating less than 5° from mathematically exact perpendicular alignment.

The term “at least partially” is intended to denote that the following property is fulfilled to a certain extent or completely.

The terms “substantially” and “essentially” are used to denote that the following feature, property or parameter is either completely (entirely) realized or satisfied or to a major degree that does not adversely affect the intended result.

The term “comprising” as used herein is intended to be non-exclusive and open-ended. Thus, for instance a composition comprising a compound A may include other compounds besides A. However, the term “comprising” also covers the more restrictive meanings of “consisting essentially of” and “consisting of,” so that for instance “a composition comprising a compound A” may also (essentially) consist of the compound A.

The various embodiments disclosed herein can be used separately and in various combinations unless specifically stated to the contrary.

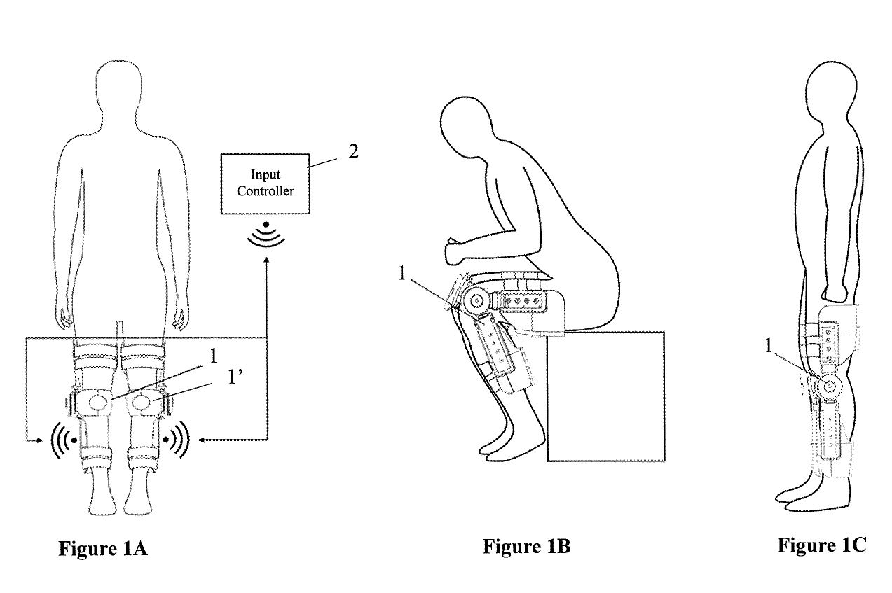

FIGS. 1A-1C illustrate views of an exemplary patient lift orthosis system attached to a patient in accordance with aspects of the present disclosure. The system comprises at least one knee orthosis 1 placed on the leg of a patient. Though the exemplary embodiment is described with reference to the leg of a human patient, the patient lift orthosis system of the present disclosure is not limited to use with the leg of a human and aspects of the present disclosure are contemplated for use on a variety of limbs for both humans and animals in need of orthosis assistance. In embodiments, the system includes two knee orthoses 1, 1′ placed on each leg of a patient. The knee orthosis 1 is centered on the patient's knees and secured to the patient via straps (discussed below). Each knee orthosis 1, 1′ includes an output controller (discussed below) that communicates via a wired and/or wireless connection with an input controller 2. In embodiments, the input controller 2 may be operated by an operator who may be a third party caregiver, the patient, and/or the input controller 2 may operate automatically pursuant to a predetermined sequence. As shown in FIG. 1B, a patient may wear the knee orthosis 1 while seated in a retracted/seated position. As shown in FIG. 1C, the input controller 2 and the output controller are configured to allow the operator to actuate the knee orthosis 1 to move the patient from the retracted/seated position to an extended/standing position. In embodiments, the input controller 2 and the output controller allow the operator to move a patient between the sitting position and the standing position, and/or vice versa.

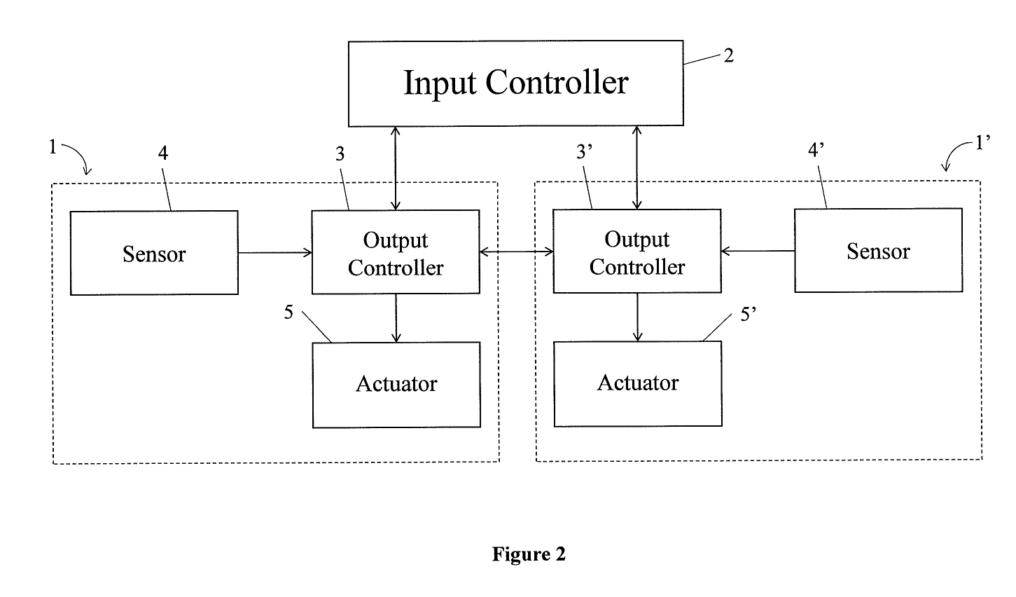

FIG. 2 illustrates a schematic view of an exemplary control system for the patient lift orthosis in accordance with aspects of the present disclosure. The control system comprises the input controller 2 and the output controller 3, 3′ of each knee orthosis 1, 1′ that are in communication (e.g., via Wi-Fi, Bluetooth, infrared, radio frequency, and/or any other wired or wireless signal transmission) with the input controller 2. In embodiments, the input controller 2 may be, e.g., a hand held remote control, a smartphone, a tablet, a smart watch, and/or another wearable technology. In embodiments, an operator may load inputs (e.g., position, speed, power, patient height, patient weight, and patient strength) into the input controller 2 by, e.g., pressing a button, touch sensors, movement or gestures, and/or via voice control.

In embodiments, the control system includes two knee orthoses 1, 1′ and the output controllers 3, 3′ of each knee orthoses 1, 1′ are configured to communicate with each other. In alternative embodiments, the control system includes a single knee orthosis 1. In certain embodiments, each output controller 3, 3′ may include a microprocessor and/or an actuator controller (not shown). Referring to FIG. 2, the control system may further include at least one sensor 4, 4′ and an actuator 5, 5′ that are each in communication with the respective output controller 3, 3′ of the knee orthoses 1, 1′.

The output controllers 3, 3′ are configured to receive and process inputs (e.g., position, speed, power, patient height, patient weight, and/or patient strength) from the input controller 2 and to actuate the actuators 5, 5′ of the knee orthoses 1, 1′ based upon the received inputs to control, e.g., a direction of lift, a desired lift speed, a patient effort level, a patient height, a patient weight, and/or a patient strength. In embodiments, the output controller 3, 3′ sends a signal to the actuators 5, 5′ by varying electrical signals and/or an amount of power routed from a power source (not shown) to the actuator 5, 5′ to achieve the desired output (e.g., position, speed, direction, power) to extend and/or retract the knee orthoses 1, 1′. By implementing aspects of the disclosure, the knee orthoses 1, 1′ may lift the patient from a sitting to a standing position and/or lower the patient from the standing to the sitting position.

According to aspects of the present disclosure, the output controllers 3, 3′ further receive sensory feedback from the at least one sensor 4, 4′ (e.g., a position sensor, an emergency stop, a limit switch, strain gages, a hall effect sensor, and/or a pressure sensor) provided on the knee orthoses 1, 1′. The output controllers 3, 3′ are configured to process the sensory feedback, independently and/or together with the input received from the input controller 2, to compare the sensory feedback to an expected response of the knee orthosis 1, 1′ based on, e.g., the patient's height, weight, strength, and level of effort to the actual response based on the strain, position, pressure, etc. In embodiments, the control system adjusts the power output of the actuator 5, 5′ accordingly throughout use to self-correct (such as, e.g., via a feedback loop) and achieve a desired output of the knee orthoses 1, 1′ (e.g., a final position of the knee orthoses 1, 1′ when a leg is provided thereon). In embodiments, the output controllers 3, 3′ are further configured to communicate with each other to share sensory feedback and to ensure that the two knee orthoses 1, 1′ are working in tandem. In embodiments, the output controllers 3, 3′ communicate the status (e.g., ready to begin, in progress, stopped, error, warning, completed, etc.) of the lift or knee orthosis 1, 1′ to the operator via the controller.

The control system is configured to provide a variable amount of power to the knee orthoses 1, 1′ (i.e., from 0-100% of the power capacity of the knee orthoses 1), such that the operator may control the amount of power and/or assist the knee orthoses 1, 1′ provide to the patient wearing the system. In embodiments, the sensors 4, 4′ may measure the amount of force a patient may contribute to move the knee between the sitting and standing position (or vice versa) and the output controllers 3, 3′ may adjust the force delivered by the knee orthosis 1, 1′ based upon the measured amount of force that the patient may contribute. By implementing aspects of the disclosure, the knee orthoses 1, 1′ may assist patient rehabilitation by steadily decreasing over time the amount of mechanical assistance provided to the patient and thereby force the patient to expend more of their own energy to move between standing and sitting positions. Further, the knee orthoses 1, 1′ has the advantage of being lightweight, portable, and may be rapidly applied and/or removed by a patient and/or third party without the need to roll the patient on their side. In addition, the knee orthoses 1, 1′ allows a patient to stand from a seated position in a manner similar to a natural sitting and standing motion thereby accelerating, improving and promoting rehabilitation. Though not depicted in each of the below-described embodiments to enable more detailed views of other components of respective knee orthosis, it should be understood that aspects of the above-described control system (e.g., the input controller, the output controller, sensors, and actuator) may be used with any of the below-described knee orthosis embodiments.

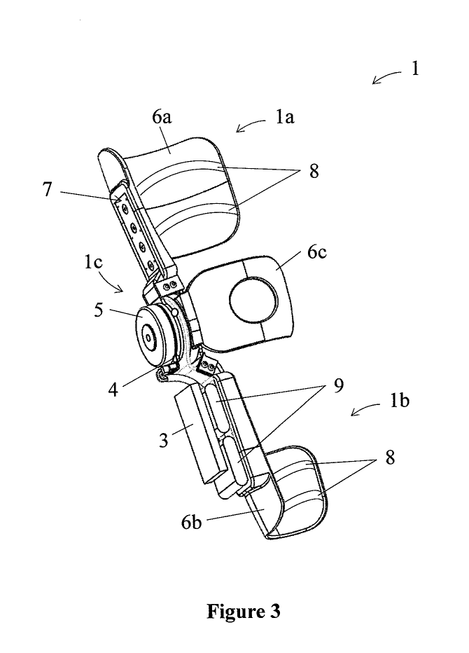

FIG. 3 illustrates a perspective view of an exemplary knee orthosis 1 of the patient lift orthosis system in accordance with aspects of the present disclosure. The knee orthosis 1 comprises the output controller 3, the at least one sensor 4, and the actuator 5, as described in detail above. The knee orthosis 1 further comprises an upper orthosis assembly la, and a lower orthosis assembly 1b that is rotatably connected to the upper orthosis assembly 1a via a joint 1c. In embodiments, the joint 1c may be uni-centric (i.e., may rotate about a single axis) or multi-centric (i.e., may rotate about two or more axes). The knee orthosis 1 further includes a plurality of cuffs 6 (i.e., two or more), which in embodiments may include a posterior thigh cuff 6a, a posterior calf cuff 6b, and an anterior knee cuff 6c. The upper orthosis assembly 1a comprises the posterior thigh cuff 6a and an upper portion of a frame 7. The lower orthosis assembly 1b comprises the posterior calf cuff 6b and a lower portion of the frame 7. The joint 1c is provided between adjoining ends of the upper and lower portions of the frame 7.

In embodiments, the plurality of cuffs 6 may include an anterior thigh cuff (not shown) and anterior lower leg cuff (not shown) that are comprised of a flexible thermoplastic material and that may be shaped and sized to correspond to opposing posterior cuffs. The anterior thigh cuff and the anterior lower leg cuff may be rigidly attached to the knee orthosis 1 on one lateral side and may employ a quick attach and release clip comprising, e.g., a ratcheting buckle, clasp, Velcro, etc. on the other lateral side.

In embodiments, the plurality of cuffs 6 are comprised of a lightweight material such as, e.g., fiberglass, carbon fiber, Kevlar, and/or other composite material. The plurality of cuffs 6 transfer the load of the patient to the frame 7 of the knee orthosis 1. The plurality of cuffs 6 are configured to hold the weight of a patient (e.g., up to approximately 350 lbs.) at any point in the sit-to-stand movement and/or the stand-to-sit movement of the knee orthosis 1. In embodiments, the plurality of cuffs 6 may be available in a variety of sizes to fit a large range of patient's leg sizes. In further embodiments (not shown), the plurality of cuffs may be removed and replaced by a user such that the same frame and actuator may be used with the various size cuffs. In yet further embodiments (not shown), the plurality of cuffs may be size-adjustable to fit a variety of leg sizes (e.g., a variety of leg lengths, widths, etc.). By implementing aspects of the disclosure, the plurality of cuffs 6 create a smooth transfer of load from the patient to the frame 7 such that there are no discrete pressure points created on the patient's soft tissue.

In embodiments (not shown), the plurality of cuffs are covered in a disposable covering and/or a covering that can be cleaned and sanitized. By implementing aspects of the disclosure, the covering can be changed and/or sanitized between each use to reduce the risk of infectious disease spreading between patients. The covering is fixed in place so as not to cause any slipping and/or sliding of the knee orthosis on the patient during use. In embodiments, the covering is attached to the plurality of cuffs with Velcro, hook and loop, snaps, magnets, etc.

As depicted in FIG. 3, the frame 7 connects the plurality of cuffs 6 to the actuator 5. The frame 7 is configured to extend along the lateral side of the leg of a patient and to provide primary structural support for transferring force from the actuator 5 to the plurality of cuffs 6. In embodiments, the frame 7 is configured to hinge at the joint 1c provided at the location of a patient's knee when a torque is applied to the frame 7 by the actuator 5. By implementing these aspects of the disclosure, the torque from the actuator 5 can be translated, through the frame 7 and the plurality of cuffs 6, into forces simultaneously applied to the posterior of the thigh, posterior of the calf, and anterior of the knee to aid the patient to move from sitting to standing and/or from standing to sitting.

In embodiments, at least one of the plurality of cuffs 6 (e.g., the posterior thigh cuff 6b and/or the posterior calf cuff 6b) may include one or more straps 8 that strap a patient to the cuffs 6. The straps 8 may wrap around the anterior of the thigh and lower leg to secure the knee orthosis 1 in place on the patient such that the knee orthosis 1 does not slip and/or slide on the patient's leg during operation. The straps 8 may be comprised of a plastic and/or a woven material, such as, e.g., nylon, Kevlar, and/or other textile. The straps 8 may be adjustable using, e.g., Velcro and/or a hook and loop system. The straps are secured to the knee orthosis 1 with a rigid attachment (not shown) such as, e.g., via a rivet, a bolt, etc. provided at one end of the straps 8. Further, at the other end of the straps 8, a quick attach and release clip (not shown) such as, e.g., a ratcheting buckle, clasp, Velcro, etc. is provided for quick fastening of the straps 8 to the patient. In embodiments, the straps 8 may include pads.

Referring to FIG. 3, the knee orthosis 1 further includes at least one power supply 9 (e.g., at least one battery) attached to the frame 7. The power supply 9 and the actuator 5 of the knee orthosis 1 are compact and lightweight and are powerful enough to lift patients weighing e.g., up to 340 lbs. with an output torque of 13 to 56 foot-pounds applied about the joint 1c of the knee orthosis 1. In embodiments, a profile of the power supply 9 and the actuator 5 of the knee orthosis 1 extends, e.g., no more than 2 inches away from the patient's leg in any direction. Further, in embodiments a single knee orthosis 1 weighs, e.g., less than 10 lbs. In embodiments, both the output controller 3 and the power supply 9 are attached to the lower orthosis assembly 1b below the joint 1c. By implementing aspects of the disclosure, the upper orthosis assembly 1a is free of the extra weight associated with the output controller 3 and the power supply 9 and thereby the patient weight capacity that the upper orthosis assembly 1a may lift when rotated by the actuator 5 is increased.

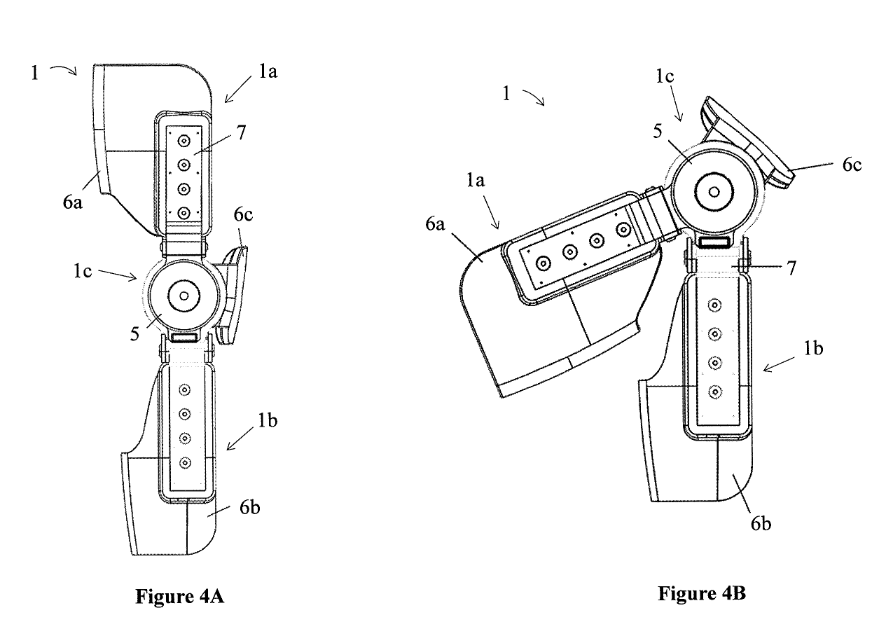

FIGS. 4A and 4B illustrate side views of an exemplary knee orthosis 1 of the patient lift orthosis system in accordance with aspects of the present disclosure. The exemplary knee orthosis 1 includes the upper orthosis assembly 1a and the lower orthosis assembly 1b, which is rotatably connected to the upper orthosis assembly 1a via the joint 1c. The knee orthosis 1 further includes the posterior thigh cuff 6a, the posterior calf cuff 6b, the anterior knee cuff 6c, the frame 7, and the actuator 5. The upper orthosis assembly 1a is configured to articulate about the joint 1c between a full extension (e.g., where the upper orthosis assembly 1a defines a 180 degree angle with the lower orthosis assembly 1b), as shown in FIG. 4A, to a full retraction (e.g., where the upper orthosis assembly 1a defines a 60 degree angle with the lower orthosis assembly 1b), as shown in FIG. 4B. The range of motion of the knee orthosis 1 mimics the range of motion of an average person such that the knee orthosis 1 does not restrict the natural sit-to-stand and stand-to-sit movement.

The knee orthosis 1 articulates by the actuator 5 applying a torque about the joint 1c. In embodiments, the anterior knee cuff 6c is configured to move independently of the frame 7, which is rigidly attached to both the posterior thigh cuff 6a and posterior calf cuff 6b. The independent movement of the anterior knee cuff 6c allows the anterior knee cuff 6c to stay at the center of the patient's knee and to maintain a comfortable force distribution on the knee. By implementing aspects of the disclosure, the movement of the knee orthosis 1 allows the patient to move from siting to standing and/or standing to sitting using natural body mechanics and the physical design of the knee orthosis 1 does not restrict that natural movement such that the system provides additional power in support the patient's natural movement. In embodiments, the knee orthosis 1 can be set by the input controller (discussed above) to limit the range of motion so that the knee orthosis 1 does not force the patient past their natural range of motion.

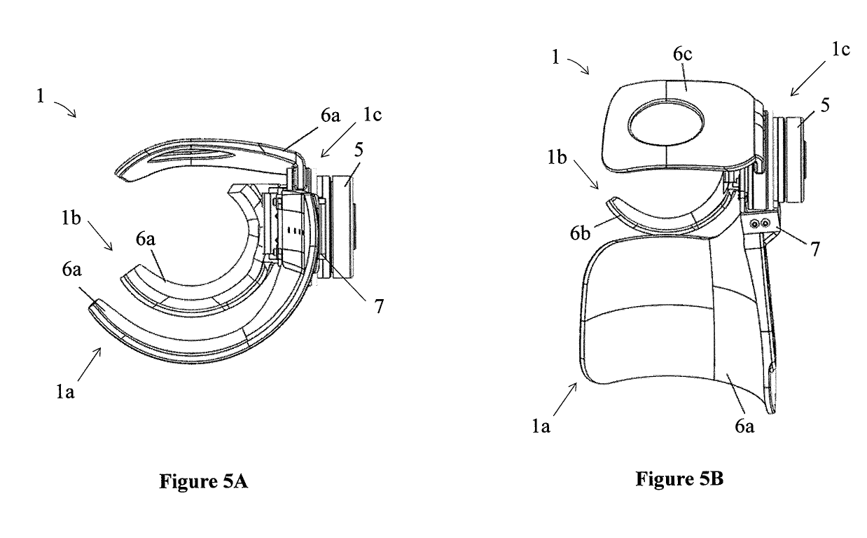

FIGS. 5A and 5B illustrate top views of an exemplary knee orthosis of the patient lift orthosis system in accordance with aspects of the present disclosure. FIG. 5A illustrates a top view of the exemplary knee orthosis in an extended position and FIG. 5B illustrates a top view of the exemplary knee orthosis in the retracted position. The exemplary knee orthosis 1 includes the upper orthosis assembly 1a and the lower orthosis assembly 1b, which is rotatably connected to the upper orthosis assembly 1a via the joint 1c. The knee orthosis 1 further includes the posterior thigh cuff 6a, the posterior calf cuff 6b, the anterior knee cuff 6c, the frame 7, and the actuator 5. The posterior thigh cuff 6a, posterior calf cuff 6b, and anterior knee cuff 6c are all attached to the frame 7 on the lateral side of the knee orthosis 1.

As depicted in FIG. 5A, a large opening is provided on the medial side of the knee orthosis 1. The large opening eases the donning and doffing of the knee orthosis 1 on the patient by permitting the leg to be placed on the knee orthosis 1 without the need to lift the leg of the patient beyond the minimal amount necessary to slide the posterior thigh cuff 6a and the posterior calf cuff 6b under the leg. In embodiments, the output controller (not shown) includes an actuator 5 control mode that permits manual rotation of the knee orthosis 1 about the joint 1c by an operator and that maintains the position of the knee orthosis 1 (e.g., by controlling power supplied to the actuator) after the manual rotation. By implementing aspects of the disclosure, the patient can be in the supine, seated, or standing position and the knee orthosis 1 can quickly and easily be donned and secured, and/or doffed and unsecured without having to roll, lift, and/or reposition the patient other than lifting his/her leg slightly to slide the posterior thigh cuff 6a and posterior calf cuff 6b under the patient's thigh and calf respectively. In addition, the angle formed by the knee orthosis 1 can be easily manipulated to match an angle of the patient's leg and ensure proper placement.

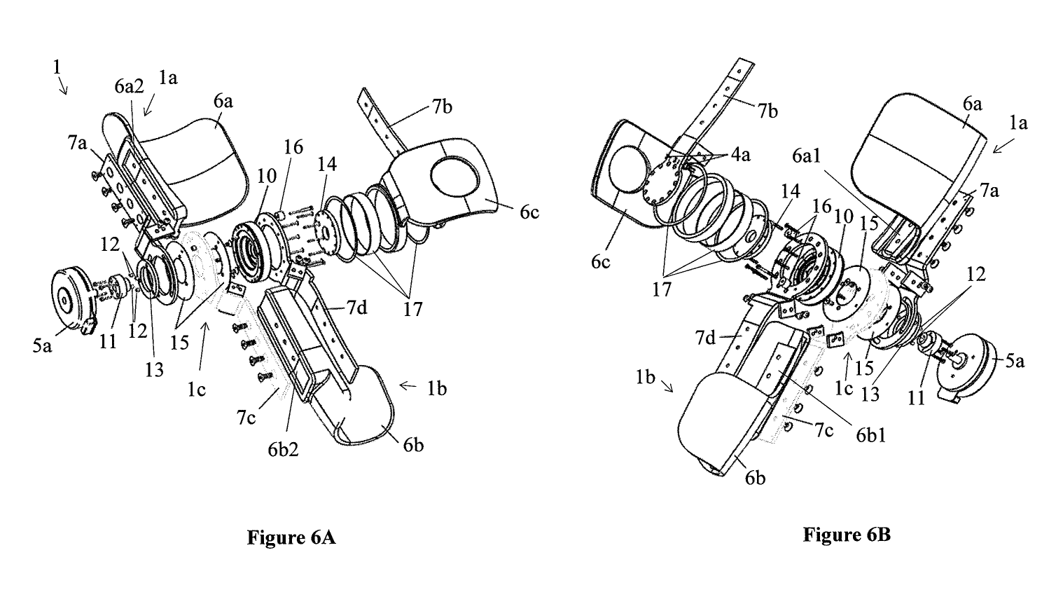

FIG. 6A illustrates an exploded perspective anterior view of an exemplary knee orthosis 1 having an electric motor 5a and a harmonic drive gearbox 10 in accordance with aspects of the present disclosure and FIG. 6B illustrates an exploded perspective posterior view of the exemplary knee orthosis of FIG. 6A. The exemplary knee orthosis 1 includes the posterior thigh cuff 6a, the posterior calf cuff 6b, the anterior knee cuff 6c, and the frame 7. The frame 7 comprises an outer thigh plate 7a, an inner thigh plate 7b, an outer calf plate 7c, and an inner calf plate 7d. In embodiments, the outer thigh plate 7a, inner thigh plate 7b, outer calf plate 7c, and inner calf plate 7d comprise plastic, steel, aluminum, titanium, other metal, and/or alloys thereof. An upper orthosis assembly 1a comprises the posterior thigh cuff 6a sandwiched between and rigidly attached to the outer thigh plate 7a and the inner thigh plate 7b. A lower orthosis assembly 1b comprises the posterior calf cuff 6b sandwiched between and rigidly attached to the outer calf plate 7c and the inner calf plate 7d. The upper orthosis assembly 1a is configured to rotate about a joint 1c relative to the lower orthosis assembly 1b.

The outer calf plate 7c and the inner calf plate 7d clamp down on the posterior calf cuff 6b to provide a rigid connection that effectively transfers load between the posterior calf cuff 6b and the frame 7. The interior surface of the posterior thigh cuff 6a, the posterior calf cuff 6b, and/or the anterior knee cuff 6c define a compound curve shape that is shaped to fit the form of a patient's leg. As shown in FIG. 6B, the shape of the inner calf plate 7d is formed to follow the form of the posterior calf cuff 6b and sits in a recessed groove 6b 1 molded into the posterior calf cuff 6b so when assembled the inner calf plate 7d sits flush against an anterior surface of the posterior calf cuff 6b. As shown in FIG. 6A, the outer calf plate 7c sits in a recessed groove 6b2 so when assembled the outer calf plate 7c sits flush with an outer surface of the posterior calf cuff 6b. As shown in FIG. 6B, the shape of the inner thigh plate 7b is similarly formed to follow the form of the posterior thigh cuff 6a and sits in a recessed groove 6a1 molded into the posterior thigh cuff 6a so when assembled the inner thigh plate 7b sits flush against an anterior surface of the posterior thigh cuff 6a. As shown in FIG. 6A, the outer thigh plate 7a sits in a recessed groove 6a2 so when assembled the outer thigh plate 7a sits flush with an outer surface of the posterior thigh cuff 6a.

An electric motor 5a (i.e., an actuator) is coupled to the harmonic drive gearbox 10 with a motor-harmonic drive coupler 11. The electric motor 5a is fixedly mounted on the outer calf plate 7c with coupler spacers 12. The knee orthosis joint 1c is provided where the upper orthosis assembly 1a and the lower orthosis assembly 1b meet and includes an outer thigh plate guide 13 that is provided between the outer thigh plate 7a and the motor-harmonic drive coupler 11 and that allows the outer thigh plate 7a to rotate relative to a housing of the electric motor 5a and outer calf plate 7c . The joint 1c further includes the harmonic drive gearbox 10, which is fixedly mounted to the inner calf plate 7d. An output of the harmonic drive gear box 10 is coupled to the inner thigh plate 7b through an inner calf plate guide 14. Accordingly, upon rotation of the electric motor 5a the output harmonic drive gear box 10 may rotate the inner thigh plate 7b and cause the upper orthosis assembly 1a to rotate relative to the lower orthosis assembly 1b about the joint 1c.

In embodiments, the electric motor 5a may be a 24 volt electric motor and the harmonic drive gearbox 10 may provide a 160:1 ratio to apply up to 56 foot-pounds of torque about the knee orthosis joint 1c so as to lift a patient weighing, e.g., up to 340 lbs.

In embodiments, thin sliding film spacers 15 comprising a low friction material are placed between the outer thigh plate 7a and the outer calf plate 7b, and between the outer calf plate 7b and the harmonic drive gearbox 10 to ease the movement of the joint 1c. Internal mechanical stop bars 16 are fixed to the inner calf plate 7d. In embodiments, the movement of the joint 1c between the upper orthosis assembly 1a and the lower orthosis assembly 1b is limited to approximately, e.g., 120 degrees by the mechanical stop bars 16 and by electrical limit switches 4a (i.e., sensors of the control system described above), which may send a stop signal to the output controller (not shown) to prevent further rotation of the electric motor 5a. By implementing aspects of the disclosure, the knee orthosis 1 may be prevented from forcing the patient's leg from moving beyond the leg's anatomical limits, thus minimizing the risk of injury.

In embodiments, the anterior knee cuff 6c surrounds the harmonic drive gearbox 10 and is sandwiched between the outer calf plate 7c and the inner calf plate 7d. The knee orthosis 1 includes a knee cuff bearing 17 disposed between the anterior knee cuff 6c and the harmonic gearbox 10 that reduces friction between the anterior knee cuff 6c, the inner calf plate 7d, outer calf plate 7c, and harmonic drive gearbox 10. The knee cuff bearing 17 allows the anterior knee cuff 6c to maintain one degree of rotational freedom and to move smoothly and independently of the upper orthosis assembly 1a and the lower orthosis assembly 1b. By implementing aspects of the disclosure, when the knee orthosis 1 is placed on the patient and the anterior knee cuff 6c is centered on the patient's kneecap, the degree of rotational freedom allows the anterior knee cuff 6c to move independently of the upper orthosis assembly 1a and the lower orthosis assembly 1b and thus remain centered on the kneecap upon rotation of the upper orthosis assembly 1a.

In embodiments (not shown), the knee orthosis 1 may include a braking system and/or clutching system between the electric motor 5a and the harmonic drive gearbox 10 to brake and/or control the rotation of the electric motor 5a.

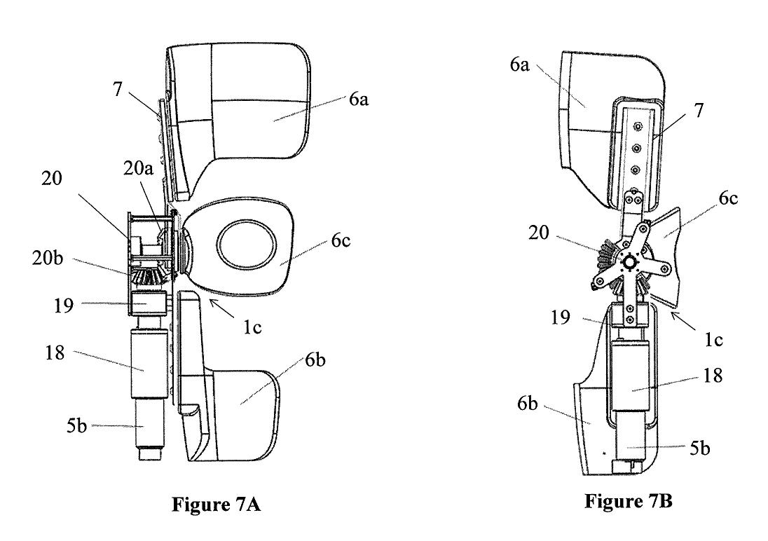

FIGS. 7A and 7B illustrate views of an exemplary knee orthosis 1 having an electric motor 5b, a high ratio gearbox 18 and a bevel gearbox 20 in accordance with aspects of the present disclosure. In embodiments, the knee orthosis includes the posterior thigh cuff 6a, the posterior calf cuff 6b, and the anterior knee cuff 6c mounted to the frame 7. The electric motor 5a is mounted to the frame 7 with the axis of the output drive shaft (not shown) substantially parallel to the patient's legs to keep the profile of the electric motor 5a low. The electric motor 5a is coupled (e.g., directly) to the high ratio gearbox 18. The output of the high ratio gearbox 18 is connected to the bevel gearbox 20 via a clutch mechanism 19. The bevel gearbox 20 has an output 20a perpendicular to an input 20b of the bevel gearbox 20. In embodiments, the output 20a of the bevel gearbox 20 can apply up to 56 foot-pounds of torque about the knee orthosis joint 1c to lift a patient weighing up to, e.g., 340 lbs.

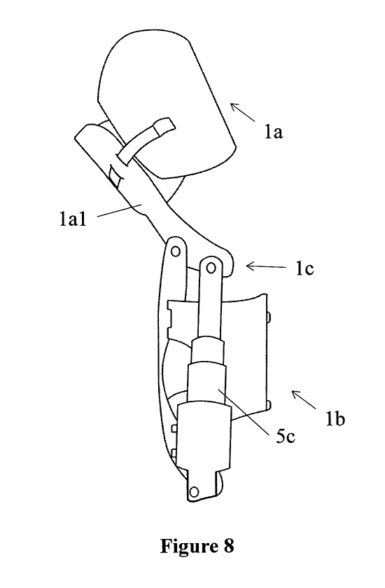

FIG. 8 illustrates a side view of an exemplary knee orthosis having a linear actuator 5c in accordance with aspects of the present disclosure. The linear actuator 5c is mounted between the upper orthosis assembly 1a and the lower orthosis assembly 1b. The upper orthosis assembly 1a and the lower orthosis assembly 1b are rotatably connected via the joint 1c that lines up with the patient's knee. The upper orthosis assembly 1a comprises a lever arm 1a1 that extends past the joint 1c and that is connected to the linear actuator 5c. The linear actuator 5c retracts and extends along an axis that is substantially parallel to the patient's leg; the retraction and extension apply a linear force on the lever arm lal that produces a requisite torque to lift a patient about the joint 1c.

In further embodiments (not shown), the knee orthosis is actuated by at least one electric motor coupled with a series of pulleys. The electric motor includes an output shaft attached to a spool and/or a drum that when wound tensions a wire to pull on a series of pulleys that compound to increase the force in the wire which terminates by anchoring into a drum and/or lever arm that produces enough output torque to lift a patient or aid in the lifting of a patient. In embodiments (not shown), a second electric motor, drum, pulley assembly is placed on the opposite side (i.e., the top or bottom) of the knee orthosis and the terminus of the wire from the first assembly and the terminus of the wire from second assembly are connected to each other and run over a drum that produces enough output torque to lift a patient or aid in the lifting of a patient. In further embodiments the electric motor may be assisted by a spring (e.g., a passive spring) that assists the motor during stand-to-sit and/or sit-to-stand movements.

In embodiments, a housing (not shown) may be provided over moving components of the actuator to shield the patient from moving components of the actuator and to prevent obstructions from interfering with the moving components.

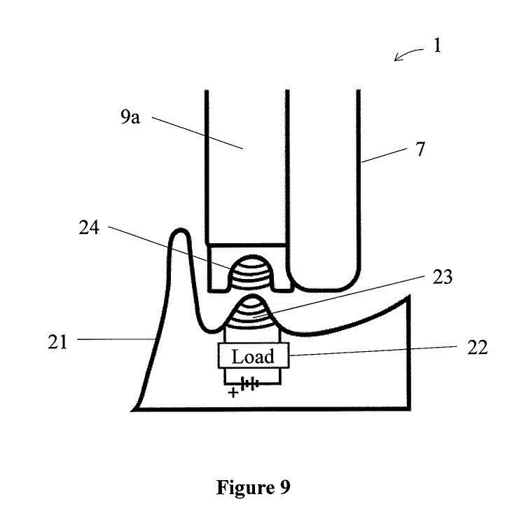

FIGS. 9 illustrates a cut-away side view of an exemplary knee orthosis 1 powered by at least one battery 9a and charged by a charging station 21 in accordance with aspects of the present disclosure. The battery 9a is mounted to the side of the frame 7 of the knee orthosis 1. In embodiments, the battery 9a may be further or alternatively mounted to posterior calf cuff (not shown) and/or the posterior thigh cuff (not shown). The battery 9a has a low profile that conforms with the shape of the knee orthosis 1 and adds, e.g., less than one inch of thickness to the profile and weighs less than, e.g., 1.5 lbs. yet is able to provide enough power to operate the knee orthosis actuator (not shown) and control system (not shown).

Referring to FIG. 9, the battery and/or batteries 9a is/are rechargeable and may be recharged by placing the knee orthosis 1 into a charging station 21. In embodiments, the charging station 21 may be wall mounted, mounted to a hospital bed, or free standing. In embodiments, the battery 9a may recharged through an induction charging system comprising a voltage load 22 and a primary coil 23 provided on the charging station 21, and a secondary coil 24 provided on the battery 9a. In embodiments, the batteries 9a may be charged in the charging station 21 via a direct contact charging system (not shown). In further embodiments, the batteries 9a may be recharged by removing the batteries and placing them in a separate charging apparatus (not shown). In still further embodiments, the batteries 9a may be recharged by plugging a power cord into the battery housing (not shown).

In embodiments, the batteries 9a are Li-Ion batteries. However, the batteries 9a are not limited to Li-Ion and may utilize any battery chemistry (or may alternatively be another portable power supply such as a fuel cell, etc.) suitable to satisfy the power and weight requirements of the knee orthosis.

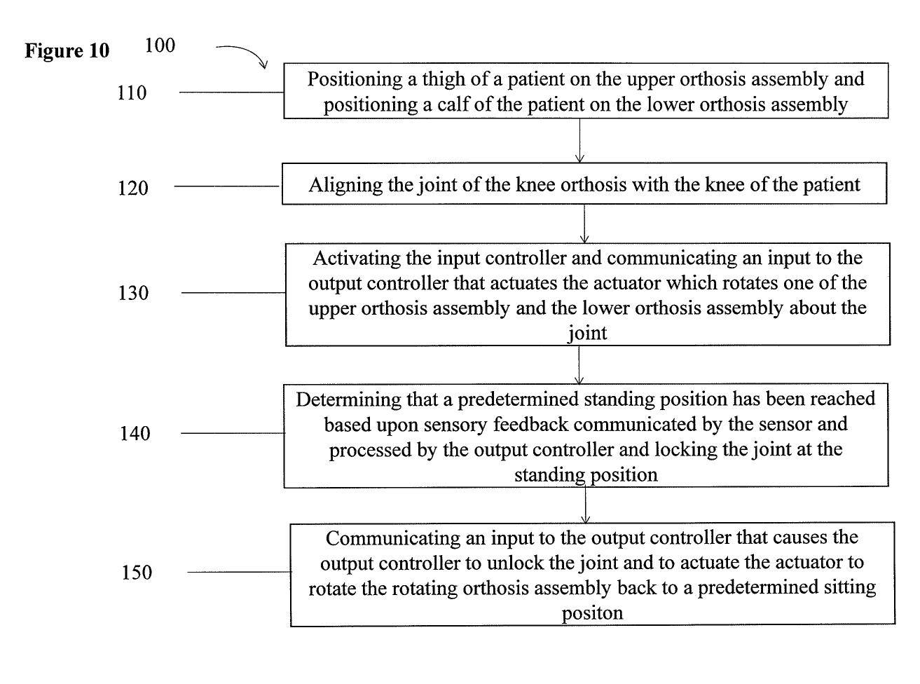

FIG. 10 illustrates an exemplary process 100 for assisting a patient to rise from a seated position to a standing position (and/or vice versa) using the patient lift orthosis system (described above). As illustrated in FIG. 10, at step 110, an operator (e.g., a nurse/caregiver and/or the patient) positions a thigh of the patient on the upper orthosis assembly (e.g., places the bottom of the thigh into a cuff and straps the patient into the cuff) and positions a calf of the patient on the lower orthosis assembly (e.g., places the bottom of the calf into a cuff and straps the patient into the cuff). In embodiments, the knee orthosis may be attached to the patient when the patient is lying supine without the need to lift the leg any significant distance (e.g., no further than necessary to slip the cuffs of the orthosis under the leg). At step 120, the operator aligns the joint of the knee orthosis with the knee of the patient. At step 130, the operator activates the input controller which communicates an input to the output controller that actuates the actuator to rotate one of the upper orthosis assembly and the lower orthosis assembly (hereinafter the rotating orthosis assembly) about the joint. The rotating orthosis assembly applies a force to the patient to lift the patient from a sitting position to a predetermined standing position. That is, the knee orthosis delivers a force to a patient's thigh and calf to move the patient's knee from a flexed to an extended position, lifting the patient from sitting to standing positions (or vice versa). In embodiments, the orthosis may measure the amount of force a patient may contribute to move the knee between the sitting and standing position (or vice versa) and may adjust the force delivered by the orthosis based upon the measured amount of force that the patient may contribute.

At step 140, the output controller determines that the predetermined standing position (e.g., which may be inputted by the input controller) has been reached based upon sensory feedback from the sensor of a change in an angle of the rotating orthosis assembly relative to the other orthosis assembly, and locks the joint (e.g., by controlling power supplied to the actuator) at the standing position. At step 150, the operator communicates an input to the output controller and the output controller unlocks the joint (e.g., by controlling power supplied to the actuator) to rotate the rotating orthosis assembly back to an angle of a predetermined sitting position (e.g., which may be inputted by the input controller). In embodiments, the process 100 for assisting a patient to rise from a seated position to a standing position using the patient lift orthosis system may utilize at least two knee orthoses that are respectively attached to the each leg of a patient.

Aspects of embodiments of the present disclosure (e.g., the input controller 2, the output controllers 3, 3′, the sensors 4, 4′, etc.) can be implemented by such special purpose hardware-based systems that can perform the specified functions or acts, or combinations of special purpose hardware and computer instructions and/or software, as described above. The control systems may be implemented and executed from either a server, in a client server relationship, or they may run on a user workstation with operative information conveyed to the user workstation. In an embodiment, the software elements include firmware, resident software, microcode, etc.

Any combination of one or more computer usable or computer readable medium(s) may be utilized. The computer-usable or computer-readable medium may be, for example, but not limited to, an electronic, magnetic, optical, electromagnetic, infrared, or semiconductor system, apparatus, device, or propagation medium. More specific examples (in the form of a non-exhaustive list) of the computer-readable medium would include the following:

an electrical connection having one or more wires,

a portable computer diskette,

a hard disk,

a random access memory (RAM),

a read-only memory (ROM),

an erasable programmable read-only memory (EPROM or Flash memory),

an optical fiber,

a portable compact disc read-only memory (CDROM),

an optical storage device,

a transmission media such as those supporting the Internet or an intranet,

a magnetic storage device

a usb key, and/or

a mobile phone.

In the context of this document, a computer-usable or computer-readable medium may be any medium that can contain, store, communicate, propagate, or transport the program for use by or in connection with the instruction execution system, apparatus, or device. The computer-usable medium may include a propagated data signal with the computer-usable program code embodied therewith, either in baseband or as part of a carrier wave. The computer usable program code may be transmitted using any appropriate medium, including but not limited to wireless, wireline, optical fiber cable, RF, etc.

Computer program code for carrying out operations of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network. This may include, for example, a local area network (LAN) or a wide area network (WAN), or the connection may be made to an external computer (for example, through the Internet using an Internet Service Provider). Additionally, in embodiments, the present invention may be embodied in a field programmable gate array (FPGA).

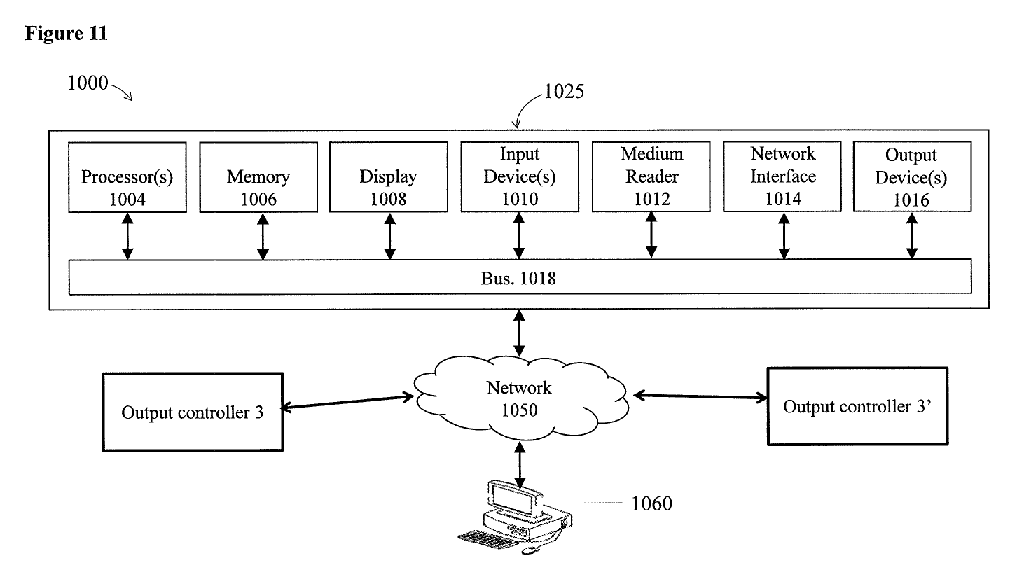

FIG. 11 is an exemplary system for use in accordance with the embodiments described herein. The system 1000 is generally shown and may include a computer system 1025 (i.e., the input controller 2, output controllers 3, 3′, etc. as discussed above), which is generally indicated. The computer system 1025 is connected to other systems or peripheral devices (i.e., the input controller 2, output controllers 3, 3′, etc. as discussed above) via, e.g., a network 1050 and/or direct connections (not shown). In embodiments, the computer system 1025 may be the input controller 2 that is connected to at least one of the output controllers 3, 3′ of the knee orthoses 1, 1′ (described above). In embodiments, the computer system 1025 may include, or be included within, any one or more computers 1060, servers, systems, communication networks or cloud environment.

The computer system 1025 may operate in the capacity of a server in a network environment, or in the capacity of a client user computer in the network environment. The computer system 1025, or portions thereof, may be implemented as, or incorporated into, various devices, such as a personal computer, a tablet computer, a set-top box, a personal digital assistant, a mobile device, a palmtop computer, a laptop computer, a desktop computer, a communications device, a wireless telephone, a personal trusted device, a web appliance, or any other machine capable of executing a set of instructions (sequential or otherwise) that specify actions to be taken by that device. Further, while a single computer system 1025 is illustrated, additional embodiments may include any collection of systems or sub-systems that individually or jointly execute instructions or perform functions.

As illustrated in FIG. 11, the computer system 1025 may include at least one processor 1004, such as, for example, a central processing unit, a graphics processing unit, or both. The computer system 1025 may also include a computer memory 1006. The computer memory 1006 may include a static memory, a dynamic memory, or both. The computer memory 1006 may additionally or alternatively include a hard disk, random access memory, a cache, or any combination thereof Of course, those skilled in the art appreciate that the computer memory 1006 may comprise any combination of memories or a single storage.

As shown in FIG. 11, the computer system 1025 may include a computer display 1008, such as a liquid crystal display, an organic light emitting diode, a flat panel display, a solid state display, a cathode ray tube, a plasma display, or any other display. The computer system 1025 may include at least one computer input device 1010, such as a keyboard, a remote control device having a wireless keypad, a microphone coupled to a speech recognition engine, a camera such as a video camera or still camera, a cursor control device, or any combination thereof. Those skilled in the art appreciate that various embodiments of the computer system 1025 may include multiple input devices 1010. Moreover, those skilled in the art further appreciate that the above-listed, exemplary input devices 1010 are not meant to be exhaustive and that the computer system 1025 may include any additional, or alternative, input devices 1010.

The computer system 1025 may also include a medium reader 1012 and a network interface 1014. Furthermore, the computer system 1025 may include any additional devices, components, parts, peripherals, hardware, software or any combination thereof which are understood as being included with or within a computer system, such as, but not limited to, an output device 1016. The output device 1016 may be, but is not limited to, a speaker, an audio out, a video out, a remote control output, or any combination thereof.

Furthermore, aspects of the disclosure may take the form of a computer program product accessible from a computer-usable or computer-readable medium providing program code for use by or in connection with a computer or any instruction execution system. The software and/or computer program product can be implemented in the environment of FIG. 11. For the purposes of this description, a computer-usable or computer readable medium can be any apparatus that can contain, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. Examples of a computer-readable storage medium include a semiconductor or solid state memory, magnetic tape, a removable computer diskette, a random access memory (RAM), a read-only memory (ROM), a rigid magnetic disk and an optical disk. Current examples of optical disks include compact disk—read only memory (CD-ROM), compact disc—read/write (CD-R/W) and DVD.

Although the present specification describes components and functions that may be implemented in particular embodiments with reference to particular standards and protocols, the disclosure is not limited to such standards and protocols. Such standards are periodically superseded by faster or more efficient equivalents having essentially the same functions. Accordingly, replacement standards and protocols having the same or similar functions are considered equivalents thereof

Accordingly, the present disclosure provides various systems, structures, methods, and apparatuses. Although the disclosure has been described with reference to several exemplary embodiments, it is understood that the words that have been used are words of description and illustration, rather than words of limitation. Changes may be made within the purview of the appended claims, as presently stated and as amended, without departing from the scope and spirit of the disclosure in its aspects. Although the disclosure has been described with reference to particular materials and embodiments, embodiments of the invention are not intended to be limited to the particulars disclosed; rather the invention extends to all functionally equivalent structures, methods, and uses such as are within the scope of the appended claims.

While the computer-readable medium may be described as a single medium, the term “computer-readable medium” includes a single medium or multiple media, such as a centralized or distributed database, and/or associated caches and servers that store one or more sets of instructions. The term “computer-readable medium” shall also include any medium that is capable of storing, encoding or carrying a set of instructions for execution by a processor or that cause a computer system to perform any one or more of the embodiments disclosed herein.

The computer-readable medium may comprise a non-transitory computer-readable medium or media and/or comprise a transitory computer-readable medium or media. In a particular non-limiting, exemplary embodiment, the computer-readable medium can include a solid-state memory such as a memory card or other package that houses one or more non-volatile read-only memories. Further, the computer-readable medium can be a random access memory or other volatile re-writable memory. Additionally, the computer-readable medium can include a magneto-optical or optical medium, such as a disk, tapes or other storage device to capture carrier wave signals such as a signal communicated over a transmission medium. Accordingly, the disclosure is considered to include any computer-readable medium or other equivalents and successor media, in which data or instructions may be stored.

Although the present application describes specific embodiments which may be implemented as code segments in computer-readable media, it is to be understood that dedicated hardware implementations, such as application specific integrated circuits, programmable logic arrays and other hardware devices, can be constructed to implement one or more of the embodiments described herein. Applications that may include the various embodiments set forth herein may broadly include a variety of electronic and computer systems. Accordingly, the present application may encompass software, firmware, and hardware implementations, or combinations thereof.

The illustrations of the embodiments described herein are intended to provide a general understanding of the various embodiments. The illustrations are not intended to serve as a complete description of all of the elements and features of apparatus and systems that utilize the structures or methods described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

Accordingly, the present disclosure provides various systems, structures, methods, and apparatuses. Although the disclosure has been described with reference to several exemplary embodiments, it is understood that the words that have been used are words of description and illustration, rather than words of limitation. Changes may be made within the purview of the appended claims, as presently stated and as amended, without departing from the scope and spirit of the disclosure in its aspects. Although the disclosure has been described with reference to particular materials and embodiments, embodiments of the invention are not intended to be limited to the particulars disclosed; rather the invention extends to all functionally equivalent structures, methods, and uses such as are within the scope of the appended claims.

One or more embodiments of the disclosure may be referred to herein, individually and/or collectively, by the term “invention” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

The above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description.

Accordingly, the novel architecture is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

While the invention has been described with reference to specific embodiments, those skilled in the art will understand that various changes may be made and equivalents may be substituted for elements thereof without departing from the true spirit and scope of the invention. While exemplary embodiments are described above, it is not intended that these embodiments describe all possible forms of the invention. Rather, the words used in the specification are words of description rather than limitation, and it is understood that various changes may be made without departing from the spirit and scope of the disclosure. In addition, modifications may be made without departing from the essential teachings of the invention. Furthermore, the features of various implementing embodiments may be combined to form further embodiments of the invention.

What is claimed is:

1. A patient lift orthosis system comprising:
an input controller; and
at least one orthosis, the at least one orthosis including:
an upper orthosis assembly;
a lower orthosis assembly rotatably attached to the upper orthosis assembly via a joint;
an output controller attached to at least one of the upper orthosis assembly, the lower orthosis assembly, and the joint, the output controller being in communication with the input controller; and
an actuator attached to at least one of the upper orthosis assembly, the lower orthosis assembly, and the joint, the actuator being in communication with the output controller, wherein
the output controller is configured to actuate the actuator to rotate at least one of the upper orthosis assembly and the lower orthosis assembly about the joint based upon an input communicated from the input controller.

2. The patient lift orthosis system according to claim 1 further comprising at least one sensor configured to provide sensory feedback, wherein
the at least one sensor is attached to at least one of the upper orthosis assembly, the lower orthosis assembly, and the joint,
the at least one sensor being in communication with the output controller, and
the output controller is configured to actuate the actuator to rotate one of the upper orthosis assembly and the lower orthosis assembly about the joint based upon the input communicated from the input controller and the sensory feedback communicated from the at least one sensor.

3. The patient lift orthosis system according to claim 1 further comprising a plurality of straps configured to hold a leg of a patient against the at least one orthosis, wherein
at least a first strap of the plurality straps is attached to the upper orthosis assembly, and
at least a second strap of the plurality of straps is attached to the lower orthosis assembly.

4. The patient lift orthosis system according to claim 1 further comprising at least one battery attached to at least one of the upper orthosis assembly and the lower orthosis assembly, wherein
the at least one battery is connected to the actuator and is configured to deliver power sufficient to rotate the one of the upper orthosis assembly and the lower orthosis assembly about the joint when the orthosis is attached to a patient.

5. The patient lift orthosis system according to claim 4, wherein
the actuator is fixedly attached to the lower orthosis assembly and rotatably attached to the upper orthosis assembly such that the actuator is configured to rotate the upper orthosis assembly about the joint relative to the lower orthosis assembly.

6. The patient lift orthosis system according to claim 5, wherein
the at least one battery and the output controller are fixed to the lower orthosis assembly.

7. The patient lift orthosis system according to claim 1, wherein
the upper orthosis assembly and the lower orthosis assembly each include at least one cuff configured to support a leg of a patient.

8. The patient lift orthosis system according to claim 7, further comprising a knee cuff that is rotatably attached to the orthosis and that is configured to rotate independently from the upper orthosis assembly and the lower orthosis assembly.

9. The patient lift orthosis system according to claim 8, wherein the at least one cuff of the upper orthosis assembly, the at least one cuff of the lower orthosis assembly, and the knee cuff are each at least one of adjustable and replaceable.

10. The patient lift orthosis system according to claim 8, wherein the at least one cuff of the upper orthosis assembly, the at least one cuff of the lower orthosis assembly, and the knee cuff each define a concave curved shape configured to receive portions of the leg of the patient.

11. The patient lift orthosis system according to claim 1, wherein
the actuator is an electric motor.

12. The patient lift orthosis system according to claim 11, wherein
the orthosis further includes a harmonic drive gearbox attached to the electric motor.

13. The patient lift orthosis system according to claim 11, wherein
the orthosis further includes a high ratio gearbox coupled to the electric motor and a bevel gearbox coupled to the high ratio gearbox via a clutch mechanism.

14. The patient lift orthosis system according to claim 1, wherein
the actuator is a linear actuator.

15. A patient lift orthosis system comprising:
an input controller; and
a first orthosis and a second orthosis, the first and the second orthoses each including:
an upper orthosis assembly;
a lower orthosis assembly rotatably attached to the upper orthosis assembly via a joint;
an output controller attached to at least one of the upper orthosis assembly, the lower orthosis assembly, and the joint, the output controller being in communication with the input controller; and
an actuator attached to at least one of the upper orthosis assembly, the lower orthosis assembly, and the joint, the actuator being in communication with the output controller; wherein
the first orthosis being configured to be attached to a first limb of a patient and the second orthosis being configured to be attached to a second limb of the patient,
for each of the first and the second orthoses respectively, the output controller is configured to actuate the actuator to rotate one of the upper orthosis assembly and the lower orthosis assembly about the joint based upon an input communicated from the input controller.

16. The patient lift orthosis according to claim 15, wherein
the output controller of the first orthosis and the output controller of the second orthosis are configured to communicate to each other.

17. The patient lift orthosis system according to claim 15 wherein
the first orthosis and the second orthosis each further include at least one sensor configured to provide sensory feedback, the at least one sensor is attached to at least one of the upper orthosis assembly, the lower orthosis assembly, and the joint, the at least one sensor being in communication with the output controller, and
for each of the first and the second orthoses respectively, the output controller is configured to actuate the actuator to rotate one of the upper orthosis assembly and the lower orthosis assembly about the joint based upon the input communicated from the input controller and the sensory feedback communicated from the at least one sensor.

18. A method of lifting a patient using a patient lift orthosis system comprising an input controller and at least one orthosis, the at least one orthosis including an upper orthosis assembly, a lower orthosis assembly rotatably attached to the upper orthosis assembly via a joint, an output controller attached to at least one of the upper orthosis assembly, the lower orthosis assembly, and the joint, the output controller being in communication with the input controller, and an actuator attached to at least one of the upper orthosis assembly, the lower orthosis assembly, and the joint, the actuator being in communication with the output controller, the method comprising:
positioning a thigh of the patient on the upper orthosis assembly and positioning a calf of the patient on the lower orthosis assembly;
aligning the joint with a knee of the patient; and
activating the input controller to communicate an input to the output controller that actuates the actuator to rotate one of the upper orthosis assembly and the lower orthosis assembly about the joint to lift the patient from a seated position to a standing position.

19. The method of lifting the patient using the patient lift orthosis system according to claim 18, further comprising locking the joint at the standing position.

20. The method of lifting the patient using the patient lift orthosis system according to claim 19 further comprising:
communicating from the input controller to the output controller to unlock the joint and to actuate the actuator to rotate the one of the upper orthosis assembly and the lower orthosis assembly about the joint to lower the patient from the standing position to the seated position.
